(19) United States
(12) Patent Application Publication
(10) Pub. No.: US 2006/0024313 A1
(43) Pub. Date: Feb. 2, 2006

(54) Agents that disrupt dimer formation in DPP-IV family of prolyl dipeptidases

(76) Inventors: Xin Chen, Miaoli (TW); Yuan-Shou Chen, Taipei City (TW)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/176,951

(22) Filed: Jul. 6, 2005

Related U.S. Application Data
(60) Provisional application No. 60/586,095, filed on Jul. 6, 2004.
(60) Provisional application No. 60/585,952, filed on Jul. 6, 2004.

(51) Int. Cl. A61K 39/395; C07H 21/04; C12P 21/06; C12N 9/64; C07K 16/40

(52) U.S. Cl. 424/146.1; 435/69.1; 435/226; 435/320.1; 435/325; 530/388.26; 536/23.2

(57) ABSTRACT

The present invention relates to the finding that C-terminal loop and propeller loop and regions thereof of the DPP-IV family of prolyl dipeptidases play an important role in dimer formation. The present invention further provides purified polypeptides comprising an amino acid sequence that mimics conserved amino acids in at least one of a C-terminal loop and a propeller loop of the dimer interface of the DPP-IV family of prolyl dipeptidases sufficient to prevent dimerization of a member of that family. Such polypeptides and other agents serve to disrupt the activity of the DPP-IV family of prolyl dipeptidases.

This application claims the benefit of U.S. Provisional Application No. 60/586,095, filed Jul. 6, 2004, and U.S. Provisional Application No. 60/585,952, filed Jul. 6, 2004, both of which are incorporated herein by reference.

### FIELD OF THE INVENTION

The present invention relates generally to peptide regions involved in dimer formation in the DPP-IV family of prolyl dipeptidases, and agents such as polypeptides that disrupt dimer formation.

### BACKGROUND OF THE INVENTION

DPP-IV (Dipeptidyl peptidase IV, also known as CD26) (E.C. 3.4.14.5) belongs to the prolyl oligopeptidase (POP) family, a subfamily of serine proteases (12,13). This class of prolyl peptidases includes DPP-IV, prolyl oligopeptidase (POP), DPP-II, DPP8, DPP9 and fibroblast activation protein (FAP) (12,13). Unlike classic serine proteases, the POP family of enzymes is highly selective towards peptides that have a proline residue at the penultimate position (18). The X-ray structures of DPP-IV and POP have shed light on the catalytic mechanisms, which differ significantly from those of the classic serine proteases, such as trypsin and subtilisin (12,14-18).

DPP-IV is a drug target for the treatment of type II diabetes (1). It is involved in the in vivo degradation of two insulin-sensing hormones, glucagon-like peptide-1 (GLP-1) and glucose-dependent insulinotropic polypeptide (GIP) (2,3). Either inhibiting the enzymatic activity of DPP-IV in various animal models or knocking out DPP-IV in mice and rats prolongs the half-lives of these two insulin-sensing hormones, increases insulin secretion and improves glucose tolerance (4-11). Hence inhibition of DPP-IV may be effective in the treatment of type II diabetes. Understanding the catalytic mechanism of DPP-IV is thus important to discovering inhibitors for the treatment of the disease.

DPP-IV consists of two domains, the α/β hydrolase domain and the p-propeller domain, with the active site in between (14-17). The substrate specificity of DPP-IV is dictated by a proline-binding pocket and a Glu205-Glu206 motif at the active site (14-17). Only small size peptides are hydrolyzed by this class of enzymes due to the unique propeller structure and/or side opening substrates used to access the active site (14-17). Among the shared properties, the most apparent difference between POP and DPP-IV is the relationship of catalytic activity with respect to its quaternary structure. POP exists in solution as a monomer and is active in such a form (15). In contrast, DPP-IV is active only as a dimer or oligomer, and monomeric DPP-IV is speculated to be inactive, even though DPP-IV monomer has never been isolated and demonstrated to be inactive (14,19).

Based on the crystal structures of DPP-IV, there are two loops located in the dimer interface and proposed to be involved in dimer interaction, the C-terminal loop at the α/β hydrolase domain and the propeller loop that extends from strand 2 of the fourth blade in the β-propeller domain (14,16,17) (FIG. 1A). The C-terminal loop of DPP-IV comprises the last 50 amino acid residues with two α-helices (aa 713 to 725 and aa 745 to 763) and one β-sheet (aa 726 to 744) interacting with the same region from the other monomer across a two-fold axis (FIG. 1B). Both hydrophobic and hydrophilic interactions have been proposed to be responsible for dimer formation (12-15).

The functional importance of the loops for the enzymatic activities of DPP-IV has not been addressed.

### SUMMARY OF THE INVENTION

The present invention provides a purified polypeptide comprising an amino acid sequence that mimics conserved amino acids in at least one of a C-terminal loop and a propeller loop of the dimer interface of the DPP-IV family of prolyl dipeptidases sufficient to prevent dimerization of a member of that family. The member of the family may be DPP-IV. The conserved amino acid sequence may include one or more amino acids corresponding to Y248, F713, V724, F730, W734, Y735, or H750 of DPP-IV.

The invention further provides a purified polypeptide comprising an amino acid sequence that mimics the amino acids of the dimer interface the DPP-IV family of prolyl dipeptidases comprising amino acids that correspond to the region spanning from F713 to H750 of DPP-IV. In another embodiment, the invention provides a purified polypeptide comprising an amino acid sequence that mimics the amino acids of the dimer interface the DPP-IV family of prolyl dipeptidases comprising amino acids that correspond to the region spanning from V724 to Y735. Other regions are encompassed by the invention.

The invention further provides a purified polypeptide comprising one or more conserved C-terminal loop amino acids selected from SEQ ID NO: 5. The invention also provides a purified polypeptide comprising one or more conserved propeller loop amino acids selected from SEQ ID NO: 6.

The invention further provides an antibody that binds specifically to the purified polypeptides of the invention.

The invention also provides a nucleic acid sequence comprising a sequence selected from SEQ ID NO: 1, SEQ ID NO: 2, SEQ ID NO: 3, and SEQ ID NO: 4. The invention also provides for the use of such sequences to produce the polypeptides of the invention.

The invention also provides a method of inhibiting dimer formation of DPP-IV family of prolyl dipeptidases by introducing a purified polypeptide comprising an amino acid sequence that mimics the amino acids of the dimer interface the DPP-IV family of prolyl dipeptidases sufficient to prevent dimerization of a member of that family. In yet further embodiments, the inhibitors are selected from antibodies, peptides, chemical compounds, and peptidomimetic compounds that bind to amino acids of the dimer interface the DPP-IV family of prolyl dipeptidases and thereby prevent dimerization of a member of that family.

### BRIEF DESCRIPTION OF THE DRAWINGS

FIG. 1 shows the location of the C-terminal Loop and H750 at the dimerization interface. Two monomers of DPP-IV are illustrated with two different colors, gray and white. (A) Dimeric DPP-IV with H750; (B) Enlarged view of the C-terminal loop with H750; (C) The residues near H750 at the C-terminal loop.

FIG. 2 shows the conservation of the C-terminal loop among prolyl dipeptidases. The sequences are from the following GenBank Accession Numbers: NP_{—}001926 (DPP-IV), Q12884 (FAP), NP_{—}001927 (DPP6), AAG29766 (DPP8), AAL47179 (DPP9), and P42658 (DPP10). The conserved His (H750 for DPP-IV) is indicated by a triangle and the catalytic triads by stars, respectively. (The amino acid sequences are shown for DPP-IV (SEQ ID NO: 7), for FAP (SEQ ID NO: 8) for DPP 6 (SEQ ID NO: 9), for DPP 8 (SEQ ID NO: 10), for DPP 9 (SEQ ID NO: 11), for DPP 10 (SEQ ID NO: 12).)

FIG. 3 shows DPP-IV is dimeric in the intact cells and in vitro. (A) Chemical crosslinking in the intact cells. Lane 1: the cells treated with DTSP followed by treatment with DTT; Lane 2: the cells treated with DTSP only; Lane 3: the cells treated with DTT only; Lane 4: the cells without treatment. Purified DPP-IV proteins run in SDS-PAGE (B) and 4-20% native gel electrophoresis (C), respectively. In panels B and C, Lane 1: sDPP-IV; Lane 2: rDPP-IV; Lane 3: H750E; Lane 4: H750A.

FIG. 4 shows the gel filtration profiles of DPP-IV proteins. (A)ₛDPP-IV; (B)ᵣDPP-IV; (C)H750A; (D) H750E.

FIG. 5 shows the sedimentation velocity analysis of DPP-IV proteins. (A) sDPP-IV; (B)ᵣDPP-IV; (C)H750A; (D) H750E. The three panels in each experiment represent the trace of absorbance at 280 nm during the sedimentation, the residues of the model fitting, and the sedimentation coefficient distribution of all species.

FIG. 6 shows the analytical ultracentrifugation analysis of separated H750A proteins. (A) Dimeric H750A after gel filtration; (B) Monomeric H750A after gel filtration.

FIG. 7 shows the result of dilution experiments. (A) sDPP-IV; (B) rDPP-IV; (C) H750A. The X-axis is the concentrations of the DPP-IVs and the Y-axis is the specific activity of the protease. The concentrations of the proteins from left to right for all the panels are 1.6 nM, 3.125 nM, 6.25 nM, 12.5 nM, 25 nM, 50 nM, 100 nM and 200 nM, respectively.

FIG. 8 shows the Sedimentation Velocity Analysis of DPP-IVs in High Salt Buffer. (A)ᵣDPP-IV; (B) H750A; (C) H750E. The panels show the sedimentation coefficient distribution of all species in high salt buffer.

FIG. 9 shows the analytical ultracentrifugation analysis results of DPP 8, showing DPP 8 is a dimer.

FIG. 10 shows the analytical ultracentrifugation analysis results of hydrophobic mutant s at the C-terminal loop—F713A, V724A, F730A, W734A, and Y735A from top to bottom.

FIG. 11 shows the analytical ultracentrifugation analysis results of Y248A which is shown to be a monomer.

FIG. 12 shows the alignment of the propeller loop among the members of the DPP-IV family of prolyl dipeptidases. (The amino acid sequences are shown for DPP-IV (SEQ ID NO: 13), for FAP (SEQ ID NO: 14) for DPP 6 (SEQ ID NO: 15), for DPP 8 (SEQ ID NO: 16), for DPP 9 (SEQ ID NO: 17), for DPP 10 (SEQ ID NO: 18).)

FIG. 13 shows the analytical ultracentrifugation analysis results of F713A and F713R from top to bottom, which are shown to be monomers.

FIG. 14 shows the analytical ultracentrifugation analysis results of H750A, showing monomers did not associate to form dimers in the presence of inhibitors in the PBS Buffer.

### DESCRIPTION OF THE EMBODIMENTS

The present invention is based upon the inventors' investigation of the role of the dimer interface of DPP-IV. The inventors identified the functional elements of the highly conserved C-terminal loop and the propeller loop of DPP-IV. The quaternary structures and catalytic activities were studied and compared among endogenous DPP-IV from human semen, recombinant wild-type and mutant DPP-IVs expressed in baculoviral infected insect cells.

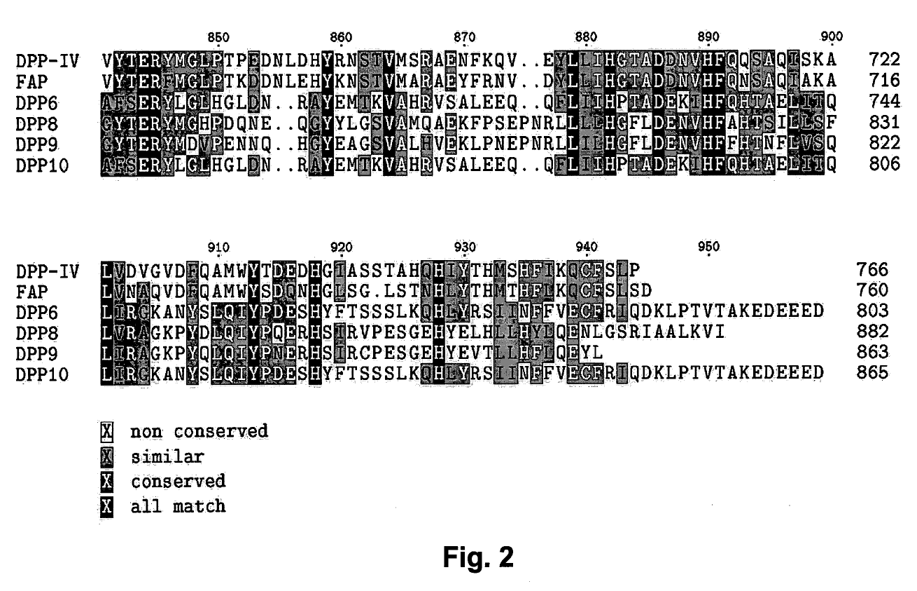

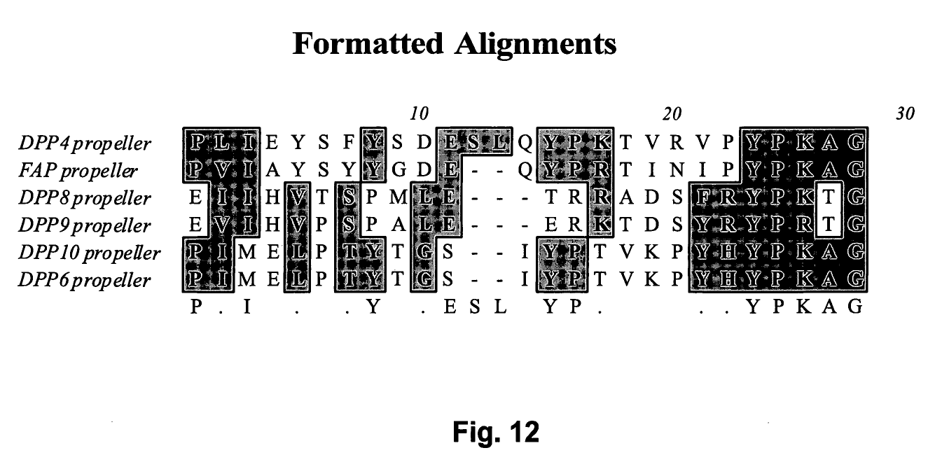

The inventors isolated a monomeric mutant DPP-IV protein altered at residue H750 of the C-terminal loop, a residue that is highly conserved among members of the DPP-IV family of prolyl dipeptidases (FIG. 2 and FIG. 12), and characterized the biochemical properties of the monomer. DPP-IV is active as a dimer, and monomeric DPP-IV has a much lower activity level. The inventors identified the C-terminal loop of DPP-IV as important for dimer formation and optimal catalysis. They found that the conserved residue H750 on the loop contributes to dimer stability.

The quaternary structures of the wild type and mutant enzymes, H750A and H750E, were determined by several independent methods including chemical crosslinking, gel electrophoresis, size exclusion chromatography, and analytical ultracentrifugation. Wild type DPP-IV exists as dimers both in the intact cell and in vitro after purification from human semen or insect cells. The H750A mutation results in a mixture of DPP-IV dimer and monomer. The H750A dimer has the same kinetic constants as those of the wild type, while the H750A monomer has 60-fold decrease in k_{cat}. Replacement of H750 with a negatively charged Glu (H750E) results in nearly exclusive monomers with a 300-fold decrease in catalytic activity. Interestingly, there is no dynamic equilibrium between the dimer and the monomer for all forms of DPP-IVs studied here.

Using similar methods, residues F713, W734, Y735, V724, and F730 of the C-terminal loop and residue Y248 of the propeller loop were also identified as important for dimer formation. The C-terminal loop, the propeller loop, as well as monomeric mutants were found to relate to the proteins' enzymatic activities.

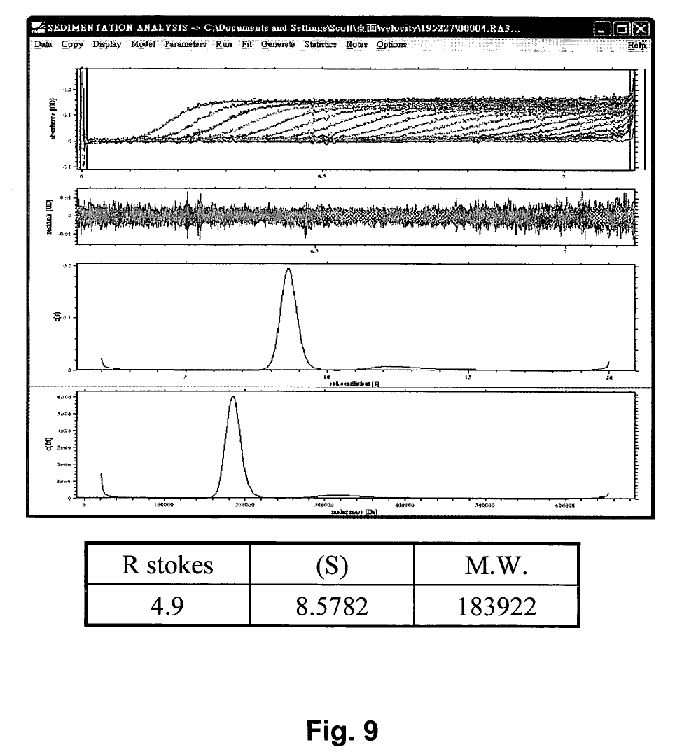

In addition, the inventors discovered that, contrary to other findings, DPP 8 exists as a dimer, as supported by the ultracentrifugation analysis result, see FIG. 9, showing sedimentation coefficient and molecular weight similar to that of a DPP-IV dimer. DPP 9 is also likely to be a dimer as it is in the same family and has the same conserved amino acid sequences as DPP 8.

Having determined the function of these residues in dimer formation in DPP-IV, the inventors noted that the dimer interface is highly conserved among DPP-IV and other prolyl dipeptidases. This family of enzymes share the properties of cleavage of the peptides after proline or imino acid residues and activity in the dimeric form, and, accordingly, the inventors have designated these enzymes as members of the DPP-IV family of proly dipeptidases. Members of the family include, but are not limited to, DPP-IV, FAP, DPP-II, DPP 8, DPP 9, DPP 10, and DPP 6.

DPP-IV and FAP are highly similar in sequence and may have arisen by gene duplication (13). FAP differs from DPP-IV in that it also has gelatinase and collagenase activity. Because of its expression sites and gelatinase/collagenase activity, FAP may have roles in cancer invasion and wound healing, and even tumorogenesis in recent studies (13).

DPP 8 shares a post-proline dipeptidyl aminopeptidase activity with DPP-IV and FAP (49). DPP 9 also has DPP-IV like peptidase activity (50).

DPP-II is another proline specific dipeptidase, often grouped together with DPP-IV. DPP-II is functionally active as a homodimer (51).

The present invention provides purified polypeptides and other agents that serve to inhibit the dimerization of DPP-IV family of prolyl dipeptidases. The purified polypeptide comprise an amino acid sequence that mimics conserved amino acids in at least one of a C-terminal loop and a propeller loop of the dimer interface of the DPP-IV family sufficient to prevent dimerization of a member of that family. The conserved amino acid sequence may include one or more amino acids corresponding to Y248, F713, V724, F730, W734, Y735, or H750 of DPP-IV. By providing polypeptides and other agents that bind to a monomer and thereby prevent dimerization, the invention provides a method to decrease the activity of the DPPj-IV family of prolyl dipeptidases.

Definitions

The terms “polypeptide,” “peptide,” and “protein,” used interchangeably herein, refer to a polymeric form of amino acids of any length, which can include naturally-occurring amino acids, coded and non-coded amino acids, chemically or biochemically modified, derivatized, or designer amino acids, amino acid analogs, peptidomimetics, and depsipeptides, and polypeptides having modified, cyclic, bicyclic, depsicyclic, or depsibicyclic peptide backbones. The term includes single chain protein as well as multimers. The term also includes conjugated proteins and fusion proteins. The term also includes peptide aptamers.

The term “purified” refers to protein substantially free of cellular material or other contaminating proteins from the cell, tissue, or body fluid sources from which the protein is derived.

The term “dimer interface” refers to a region of interaction between monomers to form dimers, comprising at least one of the C-terminal loop and the propeller loop.

The term “C-terminal loop” refers to the C-terminal region of the DPP-IV family of prolyl dipeptidases, comprising two α-helices and one β-sheet.

The term “propeller loop” refers to the region extended from the β-propeller domain of the DPP-IV family of prolyl dipeptidases.

The term “DPP-IV family of prolyl dipeptidases” refers to a subfamily of serine proteases. The members of this family are proteases that cleave proteins and peptides after the penultimate proline or imino acid residues and that are active in dimeric form. Members of the family include, but are not limited to, DPP-IV, FAP, DPP-II, DPP 8, DPP9, DPP 10, and DPP6.

The term “prevent dimerization” refers to inhibiting the formation of a dimer resulting in two monomers.

The term “mutation” refers to any change in genomic sequence, including but not limited to deletions, insertions, inversions, repeats, transitions, transversions, and type substitutions, selected according to general rules known in the art.

The term “conserved amino acid(s)” refers to amino acid(s) that have remained essentially unchanged throughout evolution and remain the same in members of the DPP-IV family of prolyl dipeptidases or amino acids subjected to typically deemed conservative substitutions by replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, and replacements between the aromatic residues Phe and Tyr.

The term “antibody” refers generally and broadly to both monoclonal and polyclonal antibodies, as well as fragments thereof. The antibodies of the invention may be chimeric, humanized, or human, using techniques standard in the art.

The term “binds specifically,” in the context of antibody binding, refers to high avidity and/or high affinity binding of an antibody to a specific epitope. Hence, an antibody that binds specifically to one epitope (a “first epitope”) and not to another (a “second epitope”) is a “specific antibody.” An antibody specific to a first epitope may cross react with and bind to a second epitope if the two epitopes share homology or other similarity.

The term “disrupting the activity” refers to a change in normal protein activities brought about by structural changes in the protein, i.e., changes in the protein's primary, secondary, tertiary, and or quaternary structures, sufficient to alter the protein's normal activities.

Function of C-terminal Loop and Propeller Loop in Dimer Formation

Dimerization is an important way to regulate the activities of many proteins, such as herpesviral and retroviral proteases, SARS 3C protease, caspase 9, and STATs (39-43). DPP-IV also forms dimers. The relationship between the quaternary structure of DPP-IV and its catalytic activity was studied. Recombinant DPP-IV and endogenous human semen DPP-IV were used. Despite a much lower extent of glycosylation, human DPP-IV expressed in insect cells has similar biochemical properties, catalytic activities and dimer structure, compared with those of the endogenous human semen DPP-IV. Using crosslinking and analytical ultracentrifugation (AUC), it was shown that DPP-IV is dimeric both in vivo and in vitro.

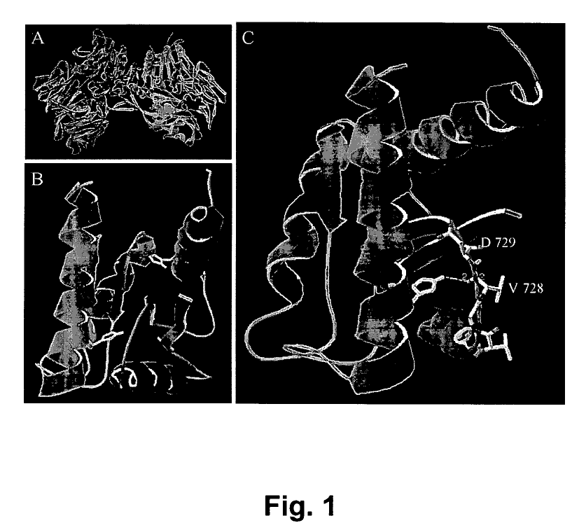

These experiments show that the C-terminal loop of DPP-IV is involved in dimer formation and is important for optimal catalytic efficiency. As the dimer interface formed by the C-terminal loop is two-fold symmetric (FIG. 1A and 1B), a single mutation is therefore functionally equivalent to double mutations in this dimeric enzyme. (The drawing in FIG. 1 was done using the DeepView (the Swiss-PdbViewer) program version 3.7 in the website http://www.expasy.org/spdbv with the structure of DPP-IV (PDB # 1N1M).) Mutations were inserted at H750 by substituting this amino acid with Alanine, giving rise to H750A and Glutamic acid, giving rise to H750E. This is the first study where monomeric DPP-IVs, H750A and H750E, were generated, purified to homogeneity and studied.

Detailed kinetic analysis showed that monomeric H750A has 60-fold drop of the k_{cat} with no change in the Kₘ, while both k_{cat} and Kₘ of H750E are remarkably changed, with a more severe effect on k_{cat} (30-fold reduction) than Kₘ value (10-fold increment). The result is particularly interesting since it reveals that the monomers of DPP-IV have much lower activities compared to the dimeric DPP-IVs.

The difference in the Kₘ between the two monomeric DPP-IV mutant proteins, H750A and H750E, might be due to a charge effect, affecting the conformation of the active site and/or the binding of the substrate. The data also suggests that the structure of DPP-IV is sensitive to packing interactions around H750. H750 is located in the vicinity of several bulky hydrophobic residues, such as V726, V728 and F730, with the exception of the charged residue D729. The carbonyl of V728 is within hydrogen bonding distance of the imidazole ring of H750, as marked on FIG. 1C. The drastic effect of H750E on disrupting the dimeric DPP-IV to monomer might be due to charge repulsion generated between E750 (H750E) of one monomer and D729 of the other (FIG. 1C). On the other hand, generation of the monomeric H750A suggests that the interaction mediated by the imidazole ring with the neighboring residues is involved in dimer stability, further indicating the important role of this residue for the C-terminal loop.

The interaction between the C-terminal loops of DPP-IV is most likely to hold the catalytic triad and the active site in an optimal position for catalysis. The formation of the monomer upon losing the dimer interaction might result in the disorientation of the loop. Since two of the three triad residues (D708 and H740) are located on the C-terminal loop and close to the actual dimerization interface (17), the optimal alignment of the triad needed for catalysis, the conformation of the substrate binding pocket or/and the position of an oxyanion hole might be affected upon monomer formation.

The studies on dimeric HCMV (human cytomegalovirus) and HIV proteases have revealed that upon the introduction of the deletion/mutation at the dimer interface, the active site configuration is changed and a loop involved in oxyanion hole stabilization is distorted (39,44). The failure of the DPP-IV propeller loop to hold the dimer together upon the introduction of the mutation on the C-terminal loop emphasizes the importance of the C-terminal loop in dimer formation and maintenance.

The importance of C-terminal loop in dimer formation is further confirmed by additional mutations at F713, W734, Y735, V724, and F730 in the C-terminal loop of DPP-IV that resulted in monomers.

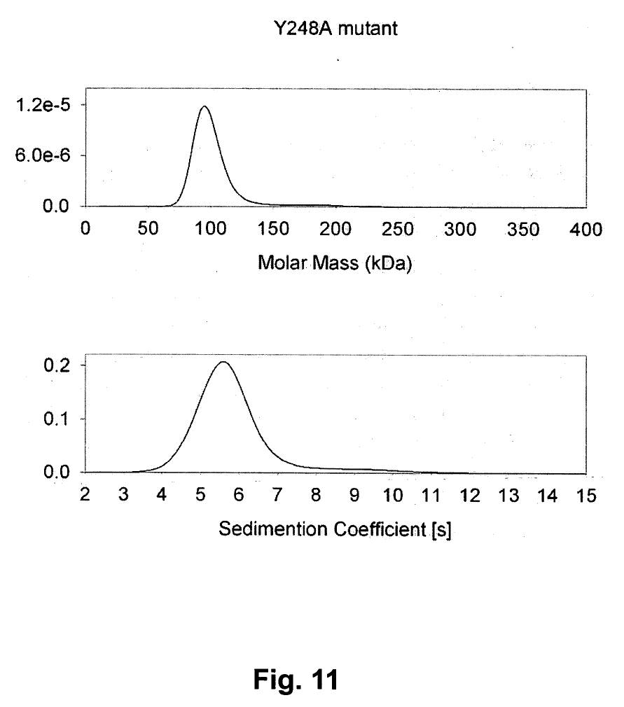

The propeller loop also contributes to dimer formation as shown by the experiment that mutations in the propeller loop can result in monomers, i.e. mutation at Y248 inhibit dimer formation of DPP-IV, see FIG. 11. Y248 is a conserved amino acid residue of the DPP-IV family of prolyl dipeptidases, see FIG. 12.

It is noted here that C-terminal loop and the propeller loop of DPP-IV are highly conserved among members of DPP-IV family of prolyl dipeptidases, (FIG. 2 and FIG. 12). This suggests that it is likely that the two loops are a general dimerization motif used by the DPP-IV family of prolyl dipeptidases.

Point Mutations Leading to Disruption of Dimer Formation

It is determined here that the identified H750, F713, W734, Y735, V724, and F730. residues, conserved among members of the DPP-IV family of prolyl dipeptidases, are involved in dimer formation. The conserved Y248 in the propeller loop of DPP-IV is another residue whose mutation can result in monomers. Other factors investigated, as described below, do not have significant effect on dimer formation.

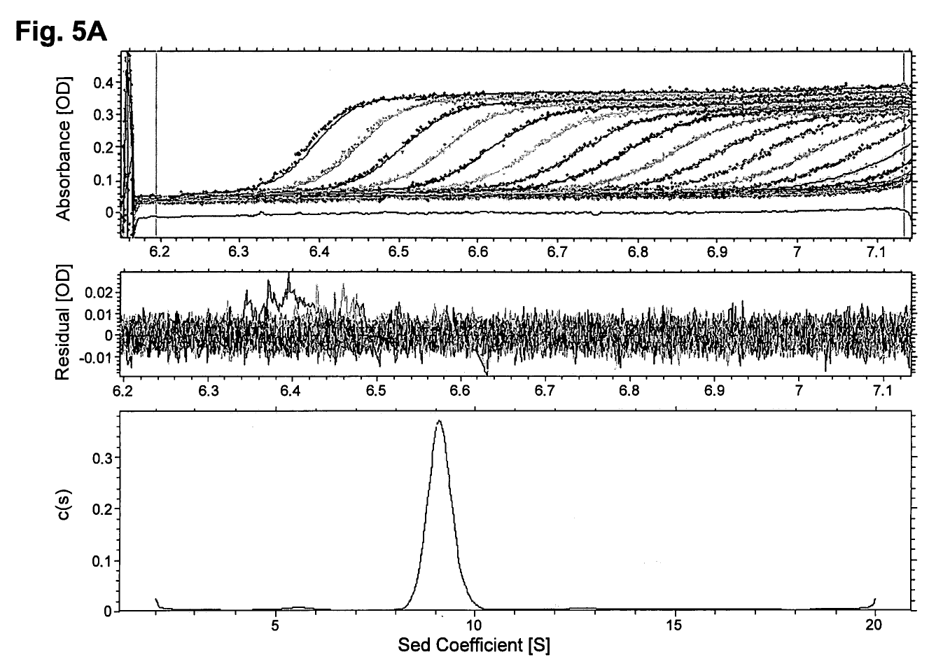

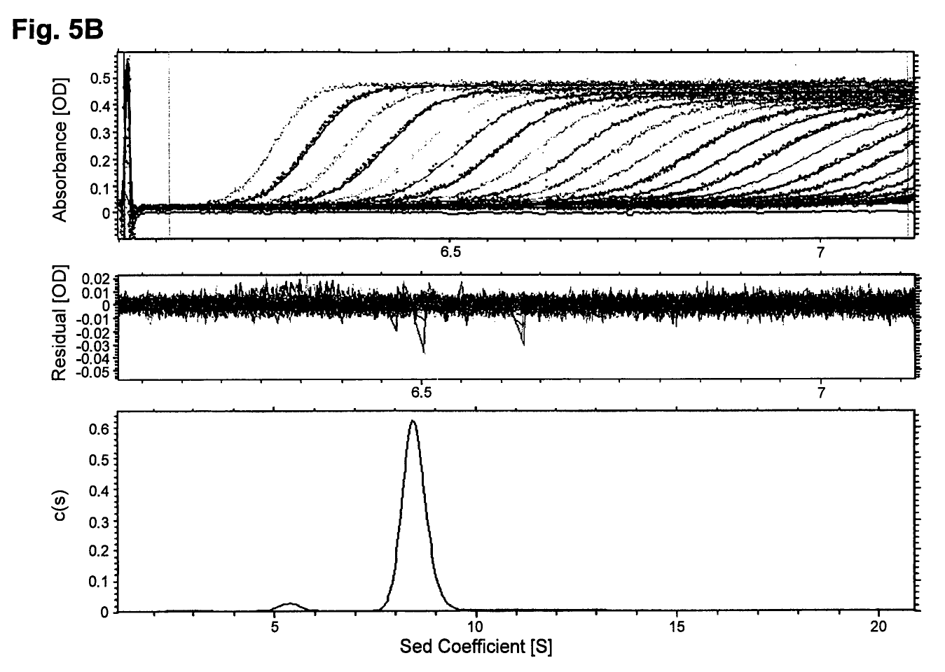

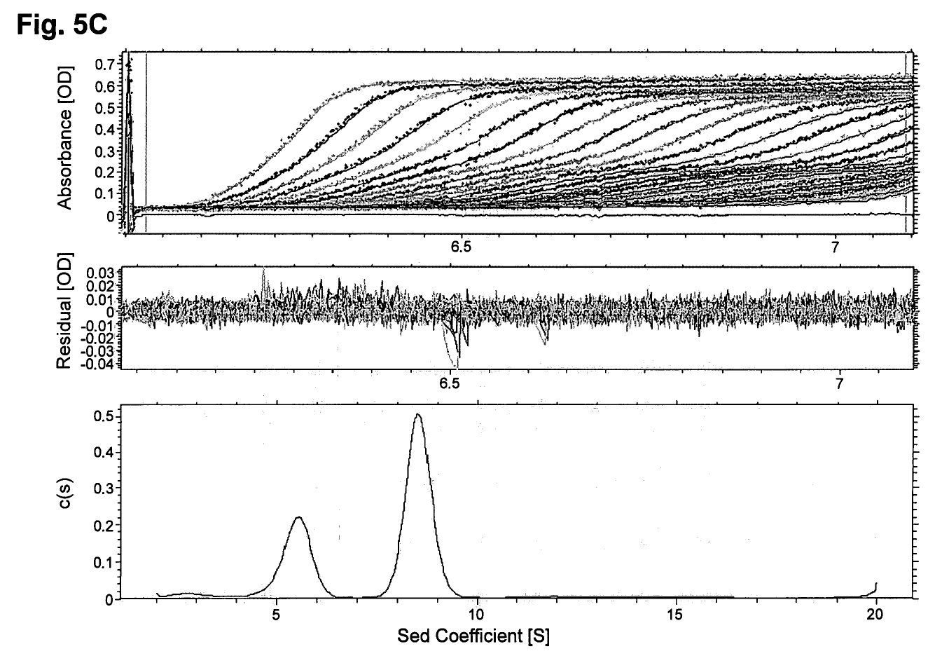

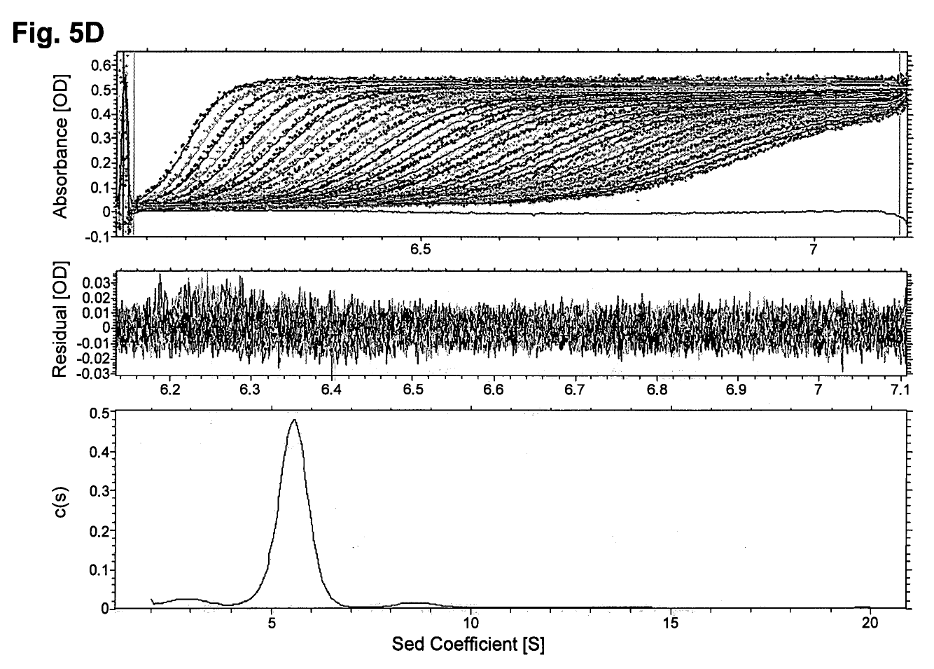

High salt concentration induces significant global conformational changes without affecting the subunit composition and the catalytic activities of the DPP-IVs. Therefore, salt has much less effect and is not capable of disrupting the dimer of DPP-IV to monomer or promoting dimer stability. This is contrary to HCMV protease, whose dimer is stabilized by high salt with a concomitant increase in catalytic activities (34,36,39,45). Based on our data (FIG. 5-8), the interaction between the monomers in DPP-IV is much stronger than that of the HCMV protease, supported by the lack of salt-induced effect for DPP-IV.

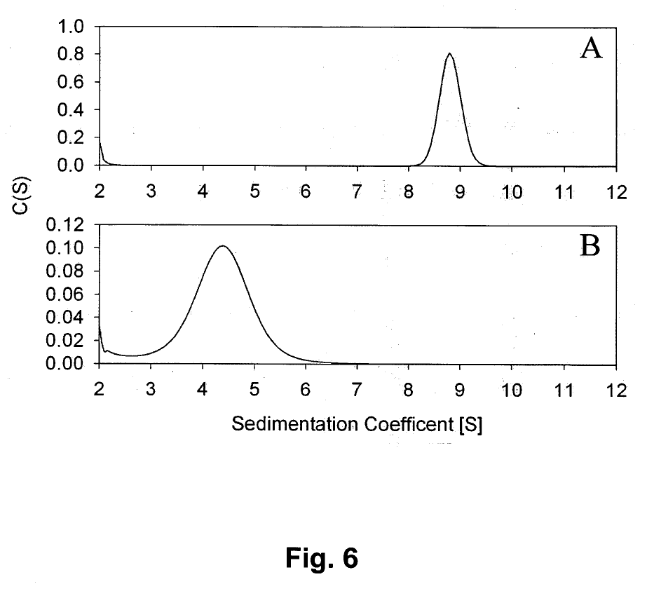

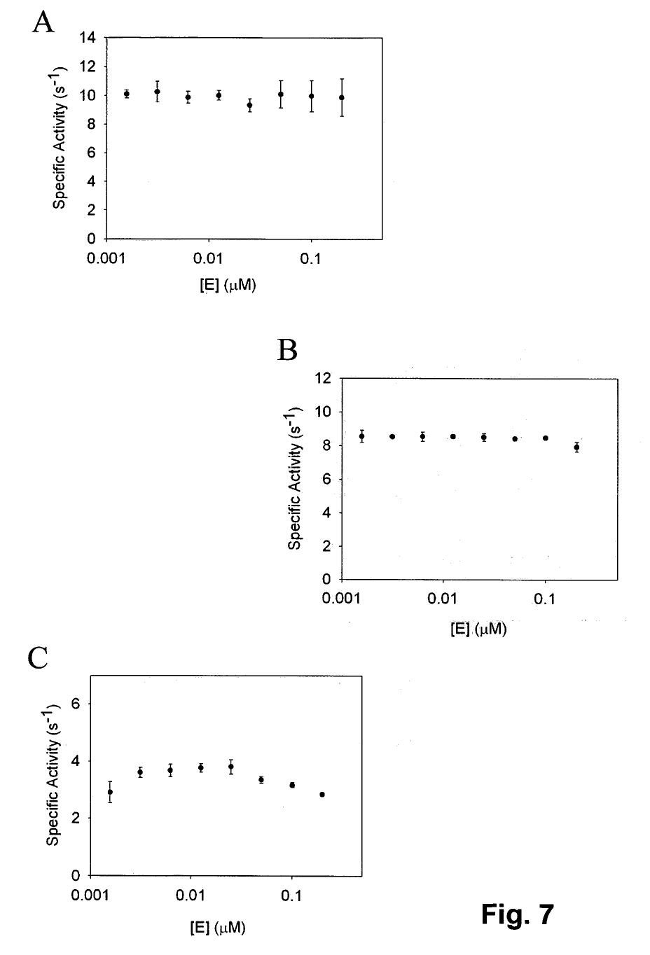

Also, there is no dynamic equilibration between the dimer and monomer of either wild type or mutant DPP-IVs in vitro (FIGS. 6 and 7). The formation of dimeric H750A by the insect cells may be assisted and promoted in vivo by chaperone proteins in the ER (endoplasmic reticulum) or by local high concentration of the proteins during synthesis. Once dimeric H750A is formed in vivo, it does not dissociate into monomer again in vitro (FIGS. 6 and 7). This indicates that there are additional interactions present in the dimer interface to compensate for the loss of the interaction by the imidazole ring of H750. The dilution experiments are consistent with the AUC experiments, indicating that there is no change of the dimer-monomer composition. This might explain the fact that up to the present time, there is no report of the isolation of the monomeric form of wild type DPP-IV.

In addition, the presence of the dipeptide product or the inhibitor failed to promote the dimerization, demonstrated in this study. These data suggested that there is not sufficient activation energy to shift either monomer to dimer or dimer to the monomer form.

Therefore, based on the experiments and analysis, the key to dimer formation lies in the dimer interface.

Conserved Dimer Interface in DPP-IV Family of Prolyl Dipeptidases

The dimer interface comprises the C-terminal loop and the propeller loop, both of which are highly conserved in the DPP-IV family of prolyl dipeptidases. It is found that certain mutations in the dimer interface, in either the C-terminal loop or the propeller loop or both, will lead to monomers and significantly reduce the activity level of the protease. The area of interest can also be a binding site for inhibiting the active dimer to disrupt the protease's activity.

Screening for Inhibitors of DPP-IV Family of Proly Dipeptidases

The present invention also provides a method of screening for inhibitors that target the dimer interface the DPP-IV family of prolyl dipeptidases. Members of the DPP-IV family of prolyl dipeptidases are drug targets for certain illnesses, e.g., DPP-IV is a drug target for immunosuppression, cancer, and type II diabetes, and FAP is an anti-cancer drug target. The dimer interface is highly conserved among members of the DPP-IV family prolyl dipeptidases and can serve as a new target site for discovering novel inhibitors that are different from the active site inhibitors.

Thus, unlike current drug discovery strategy, the present invention provides an alternative approach that focuses on finding an inhibitor that prevents dimer formation or disrupts dimer formation thus inactivating or lowering the activities of the enzyme. This approach of targeting protein-protein interaction surface presents novel binding sites and provides an alternative to the often drug-resistant active site inhibitors. Active site inhibitors are known to have more drug-resistant problems as the protein sometimes mutates to escape the drug effect, whereas the chance of such mutation escaping dimer interruption is lower. Inhibitors that may be tested include, but are not limited to, antibodies, peptides, chemical compounds, and peptidomimetic compounds.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test the invention. Further, all publications mentioned herein are incorporated by reference.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

It must be noted that, as used herein and in the appended claims, the singular forms “a,” “or,” and “the” include plural referents unless the context clearly dictates otherwise. Thus, for example, reference to “a subject polypeptide” includes a plurality of such polypeptides and reference to “the agent” includes reference to one or more agents and equivalents thereof known to those skilled in the art, and so forth.

The following examples further illustrate the invention. They are merely illustrative of the invention and disclose various beneficial properties of certain embodiments of the invention. The following examples should not be construed as limiting the invention.

### EXAMPLES

The practice of the present invention will employ, unless otherwise indicated, conventional techniques of cell biology, molecular biology, and biochemistry which are within the skill of the art. Such techniques are explained fully in the literature.

The following examples illustrate the important function of C-terminal loop of DDP-IV protein in dimer formation and certain point mutation within the C-terminal loop can disrupt dimer formation.

Experimental Procedures

Materials

The enzyme substrate H-Gly-Pro-pNA (H-Gly-Pro-p-nitroanilide) and dipeptide Gly-Pro were purchased from Bachem. Fetal bovine serum was from Hyclone. Lipofectin and the insect culture media, Grace and Express Five media, were from Invitrogen. Human liver cDNA library and linear viral vector were from Clontech. The ECL Western detection kit was from Perkin Elmer. Q Sepharose™ High Performance, CNBr-activated Sepharose 4B and Superdex 200 prepacked-columns were from Amersham-Pharmacia. The chemical crosslinker dithiobis-succinimidyl propionate (DTSP) was from Pierce. Bovine adenosine deaminase (ADA) was from Roche.

Construction of the Secreted DPP-IV Expression Plasmid

The baculovirus expression plasmid pBac8-CD5 was constructed with the secretion tag CD5. Vector pBac-PAK8 (Clontech) was modified by inserting an MT-EGFP cassette at EcoRV site to facilitate the selection of the virus expressing EGFP (20). CD5 coding sequence was amplified by PCR from human Jukat cell cDNA with the following primers: 5′-CGGGATCCATGCCCATGGGGTCTCT-3′ and 5′-CCGCTCGAGCCGAGGCAGGAAGC-3′. The CD5 cDNA fragment was released by digestion with BamH 1 and Xho I before ligation into pBac-PAK8, resulting in pBac8-CD5.

The expression plasmid of DPP-IV with the secretion tag CD5 is constructed as follows. The human cDNA fragment of DPP-IV containing amino acids 39 to 766 was amplified by PCR from a human liver cDNA library with the primers 5′-CCGCTCGAGAAAAACTTACACTCTA-3′ and 5′-GCGTCGACCTMGGTAAAGAGAAACATTG-3′, and cloned into pCR®-Blunt II-Topo vector (Invitrogen). The DPP-IV cDNA was then released by digestion with Xho I and EcoR I before ligation into the vector pBac8-CD5. Site-directed mutagenesis of DPP-IV was carried out using Pfu Turbo DNA Polymerase (Strategen). The primers used for generating H750A and H750E mutants are 5′-AGCACACCMGAAATATATACCCAC-3′ (SEQ ID NO: 1) and 5′-GTGGGTATATATTTCTTGGTGTGCT-3′ (SEQ ID NO: 2) for H750E, and 5′-AGCACACCAAGCTATATATACCCAC-3′ (SEQ ID NO: 3) and 5′-GTGGGTATATATAGCTTGGTGTGCT-3′ (SEQ ID NO: 4) for H750A, respectively. All DPP-IV cDNA fragments cloned were sequenced to verify that they contain no additional mutations other than those desired.

Insect Cell Culture, DNA Transfection, Virus Selection and Amplification

Sf9 cells were grown in Grace medium supplemented with 10% fetal bovine serum at 27° C. The transfection of DNA to Sf9 cells and the selection and amplification of the recombinant virus were carried out as described (21). For expression and purification purposes, Hi5 cells, instead of Sf9 cells, were used. Hi5 cells were infected at an M.O.I. (multiplicities of infection) of 1.0 TCID₅₀ unit/cell (TCID₅₀ is 50% tissue-culture infectious dose), determined to be the optimal condition for protein expression as described (21), and the cells were harvested at 72 hours post-transfection.

Purification of DPP-IV Proteins from Hi5 Insect Cells and Human Semen

The purification of wild-type recombinant DPP-IV was carried out as described (14). ADA affinity columns were prepared as described (22). For the purification of both H750A and H750E mutant proteins, only the ADA column was used with the omission of Triton X-100 in both the washing and elution solutions. Human semen DPP-IV protein was purified from healthy Asian male donors as described (22). The elution buffer for protein bound on an ADA column did not contain Triton X-100.

Freezing at −80° C. does not change either the quaternary structure (determined by analytical ultracentrifugation (AUC)) or the enzymatic activities of DPP-IV proteins described in this study. The purity of the protein was determined by SDS-PAGE, and proteins were visualized with Coomassie blue. The amount of protein was determined by the method of Bradford using BSA as the standard.

Polyacrylamide Gel Electrophoresis and Western Blot Analysis

Purified proteins were run on a 4-20% gradient native polyacrylamide gel with the gel running system from Amersham-Pharmacia. SDS-PAGE and Western blot analysis were conducted as described (23). Rabbit anti-DPP-IV antibody was generated in house using purified semen DPP-IV as the antigen.

Kinetic Constant Measurements

To measure the kinetic parameters, the chromogenic substrate H-Gly-Pro-pNA was utilized to initiate the reaction, which was monitored at OD 405 nm as a function of time (21). The enzyme concentrations used in the reaction were 10 nM for wild type and H750A proteins, and 100 nM for the H750E protein, respectively. The initial rate was measured with less than 10% substrate depletion for the first 10 to 300 seconds. The steady-state parameters, k_{cat} and Kₘ, were determined from initial velocity measurements at 0.5 to 5 Kₘ of the substrate concentrations. Lineweaver-Burk plots were analyzed using non-linear regression of the Michaelis-Menten equation. Correlation coefficients better than 0.99 were obtained throughout.

Chemical Crosslinking in the Intact Cells and Size Exclusion Chromatography

Chemical crosslinking in intact cells was conducted as described (24). Size exclusion chromatography was conducted at 4° C. Purified proteins (0.5 ml at a concentration of 5 uM) were applied to a Superdex 200 10/30 column (10×300-310 mm) pre-equilibrated with PBS. The sample was eluted with the same buffer at 0.3 ml/min and 0.25 ml fractions were collected. The Superdex 200 10/30 column was calibrated with the Stokes Radii of ferritin (6.1 nm), catalase (5.22 nm), aldolase (4.81 nm), albumin (3.55 nm), ovalbumin (3.05 nm) and chymotrypsinogen A (2.09 nm) from Amersham-Pharmacia.

Analytical Ultracentrifugation (AUC)

DPP-IV proteins at concentrations of around 0.1 to 0.2 mg/ml (1.2 uM to 2.3 uM) were used for AUC analysis with either PBS, high salt (100 mM Tris-HCl, 50 mM NaCl, 0.5 M Na₂SO₄, pH 7.5) or low salt (100 mM Tris-HCl, 50 mM NaCl, pH 7.5) buffers as indicated. Buffer was changed using an Amicon device and DPP-IV proteins were allowed to equilibrate for at least four hours or longer as indicated in the text at 25° C. after buffer changes. The sedimentation coefficients (S) of the enzyme were estimated by a Beckman-Coulter XL-A analytical ultracentrifuge with an An60Ti rotor as described (25). Sedimentation velocity analysis was performed at 40,000 rpm at 25° C. with standard double sector aluminum centerpieces. The UV absorption of the cells was scanned every 5 min for 4 hours. Sedimentation equilibrium was performed at 20° C. with six-channel open centerpieces and then centrifuged at 12,000 rpm for 12 hours. The data from both sedimentation velocity and sedimentation experiments were analyzed with the SedFit version 8.7 program to obtain molecular weights and sedimentation coefficients (25). Sednterp version 1.07 program is used to obtain solvent density, viscosity, Stokes' radius (Rₛ) and anhydrous frictional ratio (f/fₒ).

Dilution Experiment

Enzyme concentrations ranging from 200 nM to 1.6 nM were used in the dilution experiments. The experiments were carried out with consecutive two-fold dilutions in PBS containing 0.1% BSA and 1 mM DTT (dithiothreitol). The solution after dilution was incubated at 25° C. for 16 hours to ensure attainment of dimer-monomer equilibrium. The reaction was initiated by adding the substrate H-Gly-Pro-pNA at a final concentration of 10 uM for both wild type DPP-IV and H750A proteins. The initial rate of the reaction was recorded and converted to specific activity.

### Example 1

Human DPP-IV Protein is a Dimer in the Intact Cells and in vitro

Experiments were carried out to determine if human DPP-IV protein is a dimer in the intact cells and in vitro. From the crystal structures, human recombinant DPP-IV is shown to be a homodimer while DPP-IV purified from porcine kidney is a homotetramer (14,16,17,26). In addition, previous studies showed that purified DPP-IV proteins from various sources migrated at sizes corresponding to either dimer or tetramer/oligomer according to gel filtration experiments (19,27-30).

In Intact Cells

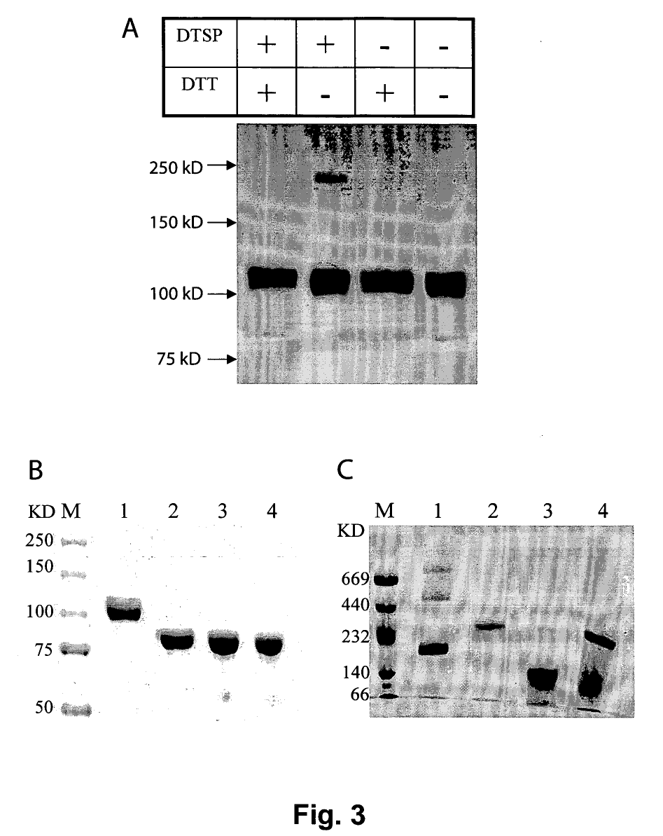

To determine the physiologically relevant oligomerization state of DPP-IV, chemical crosslinking was performed in the DPP-IV-containing Caco-2 cells. The chemical crosslinker used was DTSP, a primary amine-specific crosslinker with moderate chain length. As shown in FIG. 3A, DPP-IV could form a dimer (240 kD) in intact cells, twice the size of the monomer (approximately 120 kD) (FIG. 3A, lane 2). The crosslinker DTSP is specific since the addition of the DTT abolishes dimer formation (FIG. 3A, lane 1). The formation of dimer is DTSP-dependent since in the absence of DTSP, no dimer formation was observed (FIG. 3A lanes 3 and 4).

In Vitro

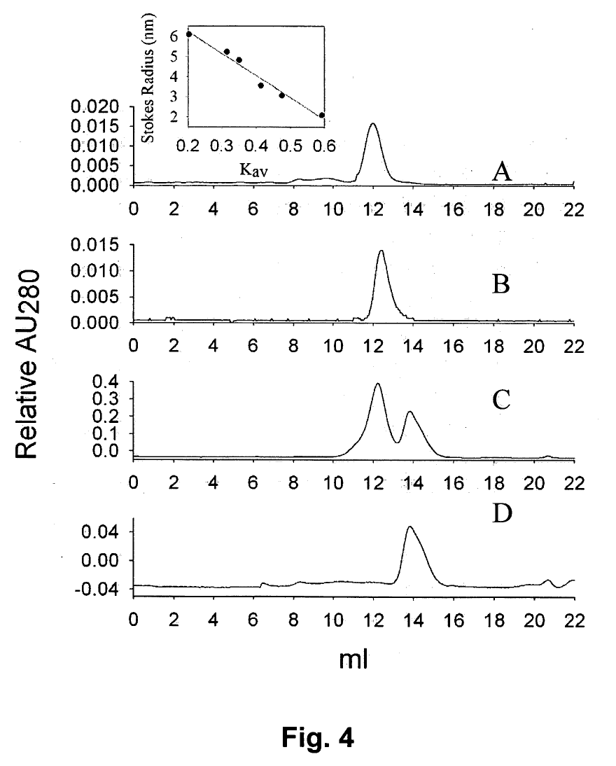

Then, a further experiment was performed to determine whether endogenous DPP-IV purified from human semen (sDPP-IV) forms dimers in vitro. The protein purified was quite pure as demonstrated by SDS-PAGE (FIG. 3B, lane 1). By measuring its kinetic constants (k_{cat} and Kₘ values), it's confirmed that purified sDPP-IV was active as reported previously (Table I) (31). On a native gel, sDPP-IV runs predominantly as a dimer of about 200 kD with the presence of minor but higher molecular weight species (FIG. 3C, lane 1). It elutes at a position corresponding to a 400 kD protein with a Stokes' radius of 5.9 nm determined by gel filtration chromatography (FIG. 4A and Table II). Calibration of the gel filtration column with the Stokes radii of the protein markers were described in the Experimental Procedures. Crosslinking of the purified protein in vitro showed that the protein is dimeric with a mass of 250 kD (data not shown).

Analytical ultracentrifugation (AUC) was used to determine the hydrodynamic properties of sDPP-IV. As shown in FIG. 5A, sDPP-IV is undoubtedly homodimeric with a sedimentation coefficient of 9.1 S (Table II), and a molecular mass of 225 kD. Notably, there is only a single peak corresponding to the dimer in the AUC experiment suggesting that dimer is the predominant form under the conditions tested. For sDPP-IV, the value of anhydrous frictional ratio f/fₒ is 1.4, indicating that the protein is non-spherical. Therefore, gel filtration does not provide an accurate measurement of sDPP-IV's quaternary structure and molecular weight, due to the protein's non-globular size. The aberrant mobility in gel filtration was also observed in previous studies with DPP-IV proteins purified from either human fibroblast cells or urine (29,32).

It is confirmed that DPP-IV is dimeric both in vivo and in vitro.

TABLE I
Kinetic constants of wild type and mutant DPP-IVs
The experiments were repeated at least three times with similar
results obtained using different batches of purified proteins. What is
shown here is one representative set of the data. Substrate used for
kinetic constant measurement is H-Gly-Pro-pNA. The experiments
were carried out as described under “Experimental Procedures.”

| | PBS buffer | | | High salt buffer | | |
|---|---|---|---|---|---|---|
| | k_{cat} | Kₘ | k_{cat}/Kₘ | k_{cat} | Kₘ | k_{cat}/Kₘ |
| | s⁻¹ | μM | μM⁻¹ s⁻¹ | s⁻¹ | μM | μM⁻¹ s⁻¹ |
| sDPP-IV | 73 | 96 | 0.76 | ND^{a} | ND | ND |
| rDPP-IV | 87 | 90 | 0.97 | 57 | 181 | 0.31 |
| H750A | 31 | 77 | 0.40 | 16 | 125 | 0.13 |
| H750A monomer | 1.4 | 64 | 0.02 | ND | ND | ND |
| H750A dimer | 73 | 79 | 0.92 | ND | ND | ND |
| H750E | 2.6 | 956 | 0.003 | 2.1 | 1092 | 0.002 |

^{a}ND, not determined.

TABLE II
Hydrodynamic properties of wild type and mutant DPP-IVs
The experiments were repeated at least twice with similar results obtained using different batches of purified proteins.
What is shown here is one representative set of the data.
The predicted monomeric Mᵣ of sDPP-IV and rDPP-IV without glycosylation is 85,246 and 84,371, respectively.

| | PBS buffer | | | | | High salt buffer | | | |
|---|---|---|---|---|---|---|---|---|---|
| | | | H750A | H750A | | | H750A | H750A | |
| | sDPP-IV | rDPP-IV | dimer | monomer | H750E | rDPP-IV | dimer | monomer | H750E |
| Stokes' radius (Rₛ) (nm) | 5.9^{a} | 5.6^{a} | 5.7^{a} | 4.6^{a} | 4.6^{a} | 4.9^{b} | 4.9^{b} | 3.7^{b} | 3.7^{b} |
| | 5.8^{b} | 5.0^{b} | 5.1^{b} | 4.1^{b} | 4.0^{b} | | | | |
| Sedimentation coefficient (s_{20, w}) (S) | 9.1 | 8.4 | 8.5 | 5.5 | 5.5 | 4.9 | 5.0 | 3.3 | 3.3 |
| Molecular weight (Mᵣ) | 225k^{c} | 187k^{c} | 186k^{c} | 98k^{c} | 98k^{c} | 154k | 158k | 78k | 79k |
| Anhydrous frictional ratio (f/fₒ) | 1.4 | 1.4 | 1.4 | 1.3 | 1.3 | ND^{d} | ND | ND | ND |

^{a}The values of the Stokes' radii were obtained from gel filtration experiments.
^{b}The values of the Stokes' radii were obtained from sedimentation velocity experiments.
^{c}The values determined were from sedimentation equilibrium experiments.
^{d}ND, not determined.

### Example 2

Properties of the Baculoviral Expressing DPP-IV Proteins

Experiments were carried out to determine whether the recombinant DPP-IV (rDPP-IV) is also dimeric in solution and has comparable biochemical properties, despite of the difference in glycosylation between the endogenous sDPP-IV and rDPP-IV. Baculoviral infected insect cells were chosen to express both wild-type and mutant DPP-IV proteins for the in vitro biochemical studies. Mutant DPP-IV was generated for determination of residues important for dimer formation.

rDPP-IV from baculoviral infected insect cells was purified and found to be as active as endogenous sDPP-IV, based on determination of k_{cat} and Kₘ values (FIG. 3B, lane 2 and Table I). The purified rDPP-IV runs at the position of around 250 kD in both native gel and gel filtration chromatography (Stokes' radius of 5.6 nm) (FIG. 3C, lane 2, FIG. 4B and Table II). Determined by velocity AUC experiments, the molecular mass of rDPP-IV is 183 kD with a sedimentation coefficient value of 8.4 S (FIG. 5B and Table II). The value of anhydrous frictional ratio (f/fₒ) is 1.4, same as that of the sDPP-IV, indicating that rDPP-IV is also a non-spherical dimer. Therefore, as demonstrated here, rDPP-IV expressed from the baculoviral infected insect cells is suitable for studying the quaternary structure and enzymatic properties of DPP-IV.

The size difference between sDPP-IV and rDPP-IV in SDS-PAGE, native gel, gel filtration and sedimentation experiments, reflects the difference in the extent and nature of the glycosylation and the non-spherical nature of the dimeric proteins. This is also consistent with the difference observed in Stokes' radii between these two wild-type proteins in AUC (Table II).

### Example 3

H750 is Important for Dimer Formation and Stability

H750 is determined to be important for dimer formation and stability through mutation and analysis of the mutant. One important interaction between two monomers of DPP-IV is provided by the C-terminal loop located at the α/β hydrolase domain (14,16,17) (FIGS. 1A and 1B). Based on the sequence alignment of the prolyl dipeptidases presented in FIG. 2, the highly conserved C-terminal loop might play an important role in the dimerization for DPP-IV and other prolyl dipeptidases. (The alignment in FIG. 2 was done using CLUSTALW and TEXSHADE programs.)

Selection for Point Mutation

There are several residues conserved in this region from aa 713 to aa 766 (SEQ ID NO: 5) (FIG. 2). H750 was chosen for mutation based on the following criteria: 1) H750 is completely conserved and intimately involved in the dimer interface based on our examination of the crystal structure; 2) compared with other conserved residues in the region, it is farthest from the triad residues D708 and H740, thus it is least likely to disturb the position of the catalytic triad if mutated.

Analysis of Mutant Proteins

H750 was mutated to Glu (H750E) to introduce an opposite charge, or Ala (H750A) to simply remove the bulky imidazole ring. Both mutant proteins were purified and found to run at the positions around 250 kD and 140 kD in the native gel (FIG. 3B and FIG. 3C, lanes 3 and 4). k_{cat} of H750A is about 3-fold smaller than that of the wild type rDPP-IV while Kₘ is not changed (Table I). Interestingly, k_{cat}/Kₘ of H750E is about 300 times smaller than that of the wild type rDPP-IV, with a 10-fold increase for Kₘ and a 30-fold decrease for k_{cat} (Table I).

The proteins were investigated to see if they remained dimeric to determine if the difference in the activities of the proteins might be correlated with their quaternary structures. First, gel filtration experiments were used to study the quaternary structures of H750A and H750E. As shown in FIG. 4C, H750A is a mixture of two peaks, with the Stokes' radius and predicted mass of 5.7 nm and 328 kD, and 4.6 nm and 144 kD, respectively. Interestingly, there is only single peak for H750E, with Stokes' radius of 4.6 nm and predicted mass of 144 kD (FIG. 4D). The Stokes' radii determined from gel filtration experiments are summarized in Table II.

Velocity AUC was thus used to analyze the quaternary structures of the mutant proteins. As shown in FIGS. 5C and 5D, as well as Table II, H750E consists only of monomer while H750A is a mixture of dimer and monomer, under the same condition as that used for the wild-type DPP-IV. The sedimentation coefficient for H750E is 5.5 S with a molecular mass of 96 kD, while H750A consists of both 5.5 S monomer and 8.5 S dimer with molecular masses of 99 kD and 188 kD, respectively (Table II). These results indicate that the H750A mutation is not enough to transform all dimer into monomer in PBS buffer which represents physiological conditions. However, the H750E mutation is sufficient to disrupt DPP-IV completely to monomers, based on AUC analysis.

Analysis and Comparison of Monomeric and Dimeric Mutant Proteins

Since H750A is a mixture, the kinetic constants of 31 s⁻¹ and 77 uM for k_{cat} and Kₘ values, respectively, were derived from both monomer and dimer forms (Table I). Thus we used gel filtration experiment to separate these two forms of H750A before subjecting them separately to sedimentation equilibrium analysis and the measurement of the enzymatic activities. As shown in FIG. 6, interestingly, both dimer and monomer maintain their subunit compositions without converting into monomer or dimer, respectively, after incubation at room temperature for up to 48 hours. This indicates that a dynamic equilibrium between dimer and monomer of H750A is extremely slow or non-existent.

The kinetic constants were measured for the monomer and the dimer of H750A after separation by gel filtration. As shown in Table I, dimeric H750A has activity similar to that of the wild type rDPP-IV indicating that, in the absence of change in quaternary structure, the mutation did not perturb the enzymatic activity. However, monomeric H750A has a 60-fold decrease in the k_{cat} but a similar Kₘ, value. Therefore, the quaternary structure of enzymes correlates with the enzymatic activities since both monomeric H750A and H750E have much lower catalytic activities compared to those of the dimeric rDPP-IV or H750A.

Mass Analysis of All Forms of DPP-IV family of Prolyl dipeptidases

Since the sedimentation velocity depends on both size and shape of the protein, sedimentation equilibrium analysis was carried out to determine unambiguously the molecular masses for all forms of DPP-IVs studied here. Summarized in Table II, for sDPP-IV, rDPP-IV, H750A dimer, H750A monomer and H750E, the molecular masses determined are 225 kD, 187 kD, 186 kD, 98 kD, 98 kD, respectively. The values obtained are comparable with those from sedimentation velocity experiments, consistent with no dynamic equilibration. All the mutant proteins analyzed have the f/fₒ values of 1.3 to 1.4, indicating that they are all non-spherical in shape.

### Example 4

Dilution Effect on Dimers

Experiments were performed to determine whether the dilution of the enzyme to very low concentration would facilitate the dissociation of the dimer into monomer, since the monomer and dimer of H750A do not equilibrate under the condition tested. The dilution method has been used to study the dimer-monomer equilibrium of several herpes viral proteases with K_{d} values in the nM range (33-35). Given that monomeric DPP-IV has very low activity as observed for monomeric H750A and H750E, dilution of the protease to a concentration near or below the K_{d} value might result in the formation of low-activity monomeric DPP-IV. As a result, the specific activity measured will be decreased (33-35). However, no decrease in specific activity were observed for either sDPP-IV or rDPP-IV even at a concentration as low as 1.6 nM (FIGS. 7A and 7B).

Similarly, the specific activity of H750A protein was also constant over the range of 200 nM to 1.6 nM (FIG. 7C). These results along with sedimentation equilibrium experiments (FIG. 6) were consistent with the lack of a dynamic equilibrium between monomeric and dimeric H750A and the small K_{d} of wild type DPP-IV dimer.

### Example 5

Effects of Salts, Active Site inhibitor and Dipeptide Product on DPP-IV Structure

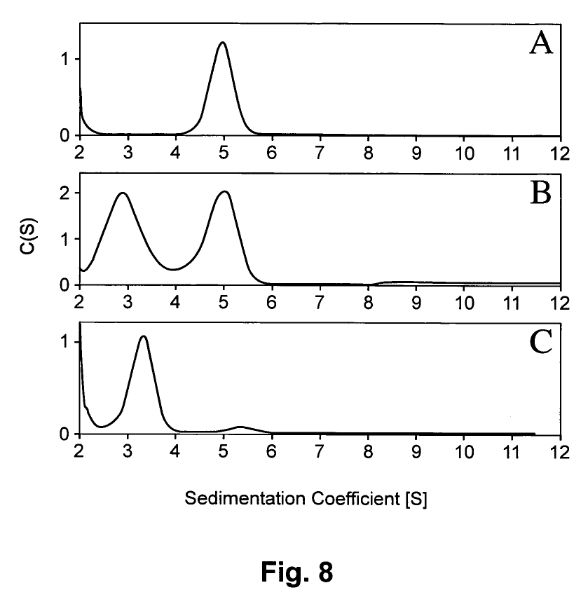

Experiments were performed to determine whether high salt concentration had any effect on the quaternary structure of DPP-IVs. High concentration of anti-chaotropic salts, such as sulfate, phosphate and citrate, enhances the stability of dimer for several herpesvirus proteases (34,36-38). High salt buffer (0.5 M sodium sulfate) has been used to probe the dimer-monomer equilibrium for dimeric HCMV protease (36). In the instant experiments, the same high salt (0.5 M sodium sulfate) was used. As shown in FIG. 8 and Table II, high salt did not change the composition of the subunits for any DPP-IV, since the molecular masses and Stokes' radii measured by sedimentation velocity still correspond to dimer, mixture of dimer-monomer and monomer for rDPP-IV, H750A and H750E, respectively, similar to results obtained in PBS buffer. However, they all show significant global conformational changes as indicated by dramatic shifts in sedimentation coefficients (FIG. 8 and Table II). The S values for the dimeric forms of rDPP-IV and H750A change from 8.4 S to 5.0 S, and the monomeric H750A and H750E from 5.5 S to 3.3 S (FIG. 8 and Table II). Interestingly, the kinetic constants of these DPP-IVs in high salt are comparable to the corresponding ones in PBS buffer with slight increment in the Kₘ for rDPP-IV and H750A (Table I). This result suggests that the interaction between the monomers of DPP-IV is quite different from that in HCMV protease. The subunit composition and activity of DPP-IVs were also studied in low salt buffer. There were no differences in either AUC analysis or catalytic activities for rDPP-IV, H750A or H750E protein, as compared to those in PBS buffer (data not shown).

### Example 6

Substrate Induce or Inhibit Dimerization

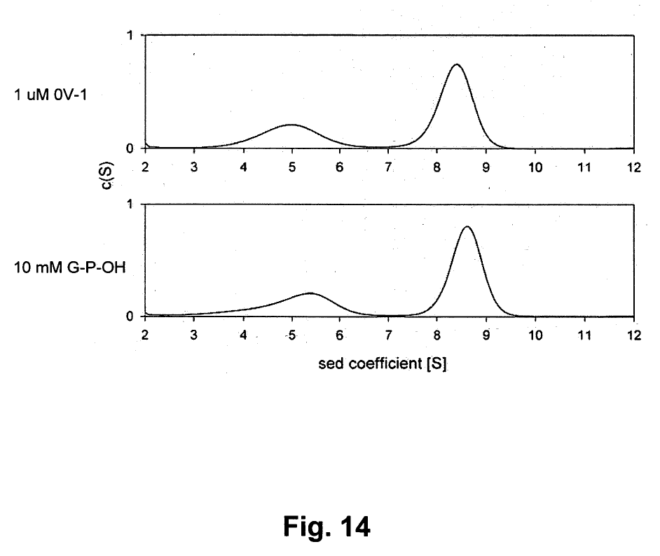

An experiment was carried out to determine whether a substrate could induce dimerization of DPP-IV. AUC was performed for H750A in the presence of the proline-mimetic inhibitor and Gly-Pro dipeptide product, since the substrate Gly-Pro-pNA is cleaved by the enzyme. The proline-mimetic inhibitor, 1-(2-amino-2-cyclohexyl-acetyl)-2-cyano-(S)-pyrrolidine, targets to the active site and has an IC₅₀ value of less than 50 nM (6). The monomeric forms of H750A and H750E did not shift to dimer in the presence of either dipeptide product or the inhibitor in either PBS or high salt buffer, see FIG. 14, showing the H750A monomers did not associate to form dimers in the presence of inhibitors OV-1 and G-P-OH in a PBS buffer.

Also, screening experiment can be carried out to determine whether a substrate could inhibit dimerization of DPP-IV prolyl dipeptidases. The goal is to find inhibitors that would prevent dimerization or disrupt dimers of DPP-IV family of prolyl dipeptidases and subsequently lower or inactivate the enzymatic activities. Proposed target sites for the inhibitors would be the dimer interface or specific amino acid sites in the dimer interface that are important for dimerization, including H750.

### Example 7

Other Sites Important for Dimer Formation and Stability

Additional mutations in the C-terminal loop were also performed at F713, W734, Y735, V724, and F730. These amino acids were substituted with Alanine resulting in mutants F713A, W734A, Y735A, V724A, and F730A respectively. These sites were shown to be important for dimerization at the C-terminal loop, with mutations at V724 and F730 having slightly less effect. Kinetic analysis of the mutants showed a much smaller K_{cat} value for the monomeric mutants, see Table III.

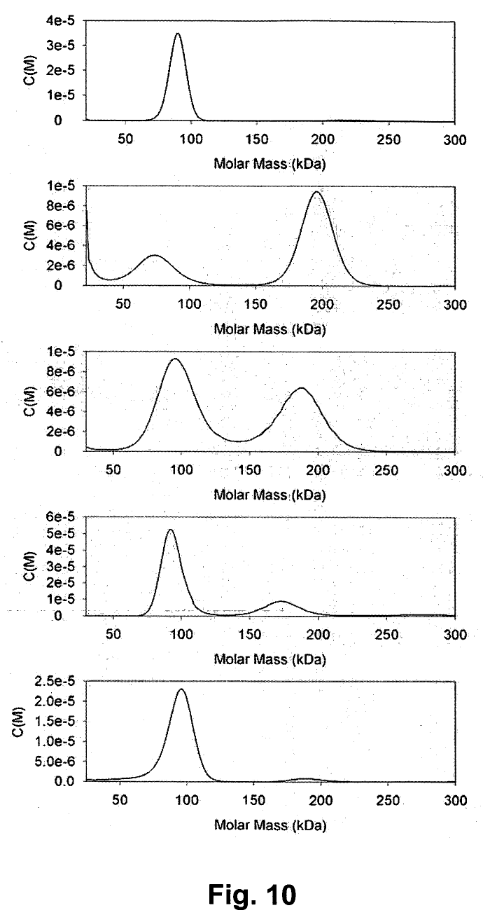

AUC analysis results on F713 (F713A), W734 (W734A), Y735 (Y735A), V724 (V724A), and F730 (F730A) are shown in FIG. 10, where F713A, W734A, and Y735A are monomers and V724A and F730A are a mixture with both monomers and dimers.

A mutation, Y248 substituted with Alanine (Y248A), in the propeller loop also disrupted the dimer and resulted in monomers, see FIG. 11. The sedimentation coefficient and molar mass were low and characteristic of monomers. Also, the K_{cat} and Kₘ value of Y248A are K_{cat}=0.01 S⁻¹, Kₘ=7 μM, which illustrated low activities compared to dimers. The propeller region is also highly conserved among members of the family of DPP-IV family of prolyl dipeptidases as shown in FIG. 12, for example aa 226-aa 252 (SEQ ID NO: 6) of the propeller region of DPP-IV. Y248 is conserved in all six proteases.

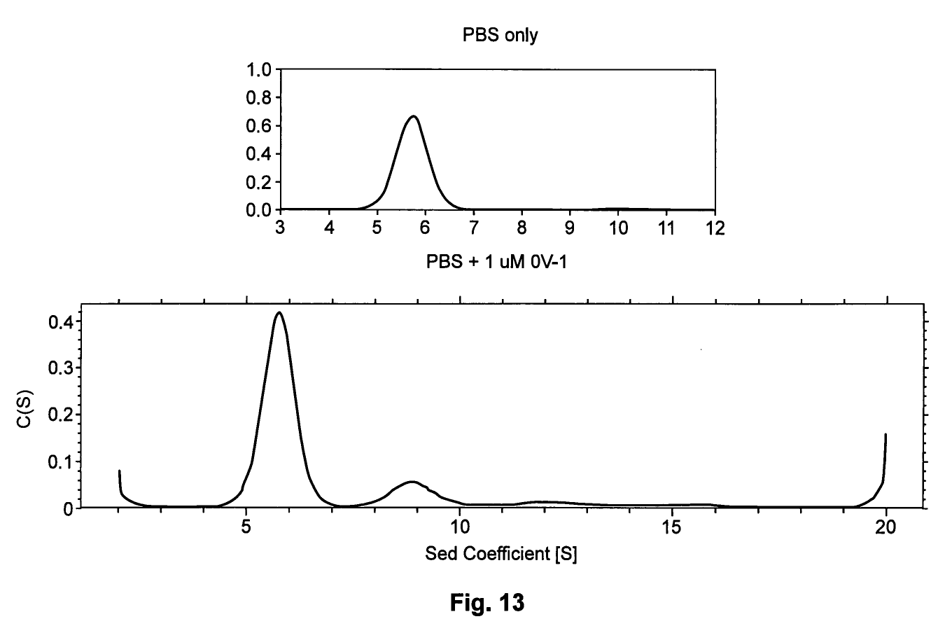

Also, mutations at F713 (substitution with Alanine (F713A) and substitution with Arginine (F713R)) resulted in monomeric DPP-IV, see FIG. 13. The F713A mutation results in monomeric DPP-IV with Km=51 uM, kcat=1 S⁻¹. The F713R mutation results in monomeric DPP-IV with Km=784 uM, kcat=2.3 S⁻¹

TABLE III
Summary of Kinetic Constants of the Mutants

| | | | | kcat/Km |
|---|---|---|---|---|
| Mutant Proteins | | kcat (s − 1) | Km (uM) | (s − 1 uM − 1) |
| 713A | monomer | 1.8 | 50.1 | 0.036 |
| 724A | Dimer | 48.3 | 87.5 | 0.55 |
| | monomer | 0.5 | 76.4 | 0.0065 |
| 730A | Dimer | 27.9 | 56.3 | 0.50 |
| | Monomer | 0.2 | 53.7 | 0.0037 |
| 734A | Monomer | 0.3 | 90.5 | 0.0033 |
| 735A | Monomer | 0.05 | 88.6 | 0.00056 |

### Example 8

Screening for Inhibitors Binding to the Dimer Interface or Regions Thereof

Fluorescence Resonance Energy Transfer (FRET) may be used in screening for inhibitors that bind to the dimer interface and inhibit dimer formation of the DPP-IV family of prolyl dippetidases.

The protein can be labeled with fluorescent protein tag, and the principle of FRET can be used to screen in a cell system for the change of fluorescence. Investigated inhibitor is added to the protein or intact cells and a conformational change from dimer to monomer can be detected by a change in fluorescence.

The candidate inhibitor may be a chemical compound, an antibody, a peptide, or a peptidomimetic compound and is selected or designed based on the dimer interface or regions thereof involved in dimerization.

### REFERENCES

The publications discussed herein and listed below are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.
1. 1. Drucker, D. J. (2003) Expert Opin Investig Drugs 12, 87-100
2. Mentlein, R. (1999) Regul Pept 85, 9-24
3. Pauly, R. P., Rosche, F., Wermann, M., McIntosh, C. H., Pederson, R. A., and Demuth, H. U. (1996) J Biol Chem 271, 23222-23229
4. Pospisilik, J. A., Stafford, S. G., Demuth, H. U., McIntosh, C. H., and Pederson, R. A. (2002) Diabetes 51, 2677-2683
5. Ahren, B., Holst, J. J., Martensson, H., and Balkan, B. (2000) Eur J Pharmacol 404, 239-245
6. Villhauer, E. B., Brinkman, J. A., Naderi, G. B., Dunning, B. E., Mangold, B. L., Mone, M. D., Russell, M. E., Weldon, S. C., and Hughes, T. E. (2002) J Med Chem 45, 2362-2365
7. Deacon, C. F., Danielsen, P., Klarskov, L., Olesen, M., and Holst, J. J. (2001) Diabetes 50, 1588-1597
8. Sudre, B., Broqua, P., White, R. B., Ashworth, D., Evans, D. M., Haigh, R., Junien, J. L., and Aubert, M. L. (2002) Diabetes 51, 1461-1469
9. Riemann, D., Kehlen, A., and Langner, J. (1995) Clin Exp Immunol 100, 277-283
10. Marguet, D., Baggio, L., Kobayashi, T., Bernard, A. M., Pierres, M., Nielsen, P. F., Ribel, U., Watanabe, T., Drucker, D. J., and Wagtmann, N. (2000) Proc Natl Acad Sci USA 97, 6874-6879
11. Conarello, S. L., Li, Z., Ronan, J., Roy, R. S., Zhu, L., Jiang, G., Liu, F., Woods, J., Zycband, E., Moller, D. E., Thornberry, N. A., and Zhang, B. B. (2003) Proc Natl Acad Sci USA 100, 6825-6830
12. Polgar, L. (2002) Cell Mol Life Sci 59, 349-362
13. Rosenblum, J. S., and Kozarich, J. W. (2003) Curr Opin Chem Biol 7, 496-504
14. Rasmussen, H. B., Branner, S., Wiberg, F. C., and Wagtmann, N. (2003) Nat Struct Biol 10, 19-25
15. Fulop, V., Bocskei, Z., and Polgar, L. (1998) Cell 94, 161-170
16. Engel, M., Hoffmann, T., Wagner, L., Wermann, M., Heiser, U., Kiefersauer, R., Huber, R., Bode, W., Demuth, H. U., and Brandstetter, H. (2003) Proc Natl Acad Sci USA 100, 5063-5068
17. Thoma, R., Loffler, B., Stihie, M., Huber, W., Ruf, A., and Hennig, M. (2003) Structure (Camb) 11, 947-959
18. De Meester, I., Durinx, C., Bal, G., Proost, P., Struyf, S., Goossens, F., Augustyns, K., and Scharpe, S. (2000) Adv Exp Med Biol 477, 67-87
19. Lambeir, A. M., Diaz Pereira, J. F., Chacon, P., Vermeulen, G., Heremans, K., Devreese, B., Van Beeumen, J., De Meester, I., and Scharpe, S. (1997) Biochim Biophys Acta 1340, 215-226
20. Lee, D. F., Chen, C. C., Hsu, T. A., and Juang, J. L. (2000) J Virol 74, 11873-11880
21. Chen, Y. S., Chien, C. H., Goparaju, C. M., Hsu, J. T., Liang, P. H., and Chen, X. (2004) Protein Expr Purif 35, 142-146
22. de Meester, I., Vanhoof, G., Lambeir, A. M., and Scharpe, S. (1996) J Immunol Methods 189, 99-105
23. Sambrook, J., Fritsch, E. F., and Maniatis, T. (1989) Molecular cloning: a laboratory manual, 2nd Ed., Cold Spring Harbor Laboratory, Cold Spring Harbor, N.Y.
24. Kreis, T. E., and Lodish, H. F. (1986) Cell 46, 929-937
25. Chang, H. C., and Chang, G. G. (2003) J Biol Chem 278, 23996-24002
26. Aertgeerts, K., Ye, S., Tennant, M. G., Kraus, M. L., Rogers, J., Sang, B. C., Skene, R. J., Webb, D. R., and Prasad, G. S. (2004) Protein Sci 13, 412-421
27. Puschel, G., Mentlein, R., and Heymann, E. (1982) Eur J Biochem 126, 359-
28. Kenny, A. J., Booth, A. G., George, S. G., Ingram, J., Kershaw, D., Wood, E. J., and Young, A. R. (1976) Biochem J 157, 169-182
29. Saison, M., Verlinden, J., Van Leuven, F., Cassiman, J. J., and Van den Berghe, H. (1983) Biochem J 216, 177-183
30. Daddona, P. E., and Kelley, W. N. (1978) J Biol Chem 253, 4617-4623
31. Lambeir, A. M., Proost, P., Durinx, C., Bal, G., Senten, K., Augustyns, K., Scharpe, S., Van Damme, J., and De Meester, I. (2001) J Biol Chem 276, 29839-29845
32. Kato, T., Hama, T., Kojima, K., Nagatsu, T., and Sakakibara, S. (1978) Clin Chem 24, 1163-1166
33. Liang, P. H., Brun, K. A., Feild, J. A., O'Donnell, K., Doyle, M. L., Green, S. M., Baker, A. E., Blackburn, M. N., and Abdel-Meguid, S. S. (1998) Biochemistry 37, 5923-5929
34. Margosiak, S. A., Vanderpool, D. L., Sisson, W., Pinko, C., and Kan, C. C. (1996) Biochemistry 35, 5300-5307
35. Darke, P. L., Cole, J. L., Waxman, L., Hall, D. L., Sardana, M. K., and Kuo, L. C. (1996) J Biol Chem 271, 7445-7449
36. Khayat, R., Batra, R., Bebemitz, G. A., Olson, M. W., and Tong, L. (2004) Biochemistry 43, 316-322
37. Schmidt, U., and Darke, P. L. (1997) J Biol Chem 272, 7732-7735
38. Yamanaka, G., Dilanni, C. L., O'Boyle, D. R., 2nd, Stevens, J., Weinheimer, S. P., Deckman, I. C., Matusick-Kumar, L., and Colonno, R. J. (1995) J Biol Chem 270, 30168-30172
39. Batra, R., Khayat, R., and Tong, L. (2001) Nat Struct Biol 8, 810-817
40. Darke, P. L. (1994) Methods Enzymol 241, 104-127
41. Renatus, M., Stennicke, H. R., Scott, F. L., Liddington, R. C., and Salvesen, G. S. (2001) Proc Natl Acad Sci USA 98, 14250-14255
42. Braunstein, J., Brutsaert, S., Olson, R., and Schindler, C. (2003) J Biol Chem 278, 34133-34140
43. Fan, K., Wei, P., Feng, Q., Chen, S., Huang, C., Ma, L., Lai, B., Pei, J., Liu, Y., Chen, J., and Lai, L. (2004) J Biol Chem 279, 1637-1642
44. Ishima, R., Torchia, D. A., Lynch, S. M., Gronenborn, A. M., and Louis, J. M. (2003) J Biol Chem 278, 43311-43319
45. Cole, J. L. (1996) Biochemistry 35, 15601-15610
46. Boggetto, N., and Reboud-Ravaux, M. (2002) Biol Chem 383, 1321-1324
47. Zutshi, R., Brickner, M., and Chmielewski, J. (1998) Curr Opin Chem Biol 2, 62-66
48. De Clercq, E. (2002) Nat Rev Drug Discov 1, 13-25
49. Abbott, A. C. (2000) Eur. J. Biochem 267, 6140-6150
50. Ajami, K. (2004) Biochimica et Biophysica Acta 1697, 18-28
51. Leiting B. et al. (2003) Biochem J. 371, 525-532

What is claimed is:

1. A purified polypeptide comprising an amino acid sequence that mimics conserved amino acids in at least one of a C-terminal loop and a propeller loop of a dimer interface of a DPP-IV family of prolyl dipeptidases sufficient to prevent dimerization of members of the DPP-IV family of prolyl dipeptidases.

2. The purified polypeptide of claim 1, wherein the family member is DPP-IV.

3. The purified polypeptide of claim 1, wherein the conserved amino acids are selected from Y248, F713, V724, F730, W734, Y735, and H750.

4. The purified polypeptide of claim 1, wherein the amino acid sequence correspond to a region spanning from F713 to H750 of DPP-IV.

5. The purified polypeptide of claim 4, wherein the region spans from V724 to Y735.

6. A purified polypeptide comprising one or more conserved C-terminal loop amino acids selected from SEQ ID NO: 5.

7. A purified polypeptide comprising one or more conserved propeller loop amino acids selected from SEQ ID NO: 6.

8. An antibody that binds specifically to the polypeptides of claim 1, claim 6, or claim 7.

9. A method of inhibiting dimer formation of DPP-IV family of prolyl dipeptidases by introducing the polypeptide of claim 1, claim 6 or claim 7.

10. A method of inhibiting dimer formation of DPP-IV family of prolyl dipeptidases by introducing an inhibitor selected from antibodies, peptides, chemical compounds, and peptidomimetic compounds, wherein the inhibitor mimics conserved amino acids in at least one of a C-terminal loop and a propeller loop of a dimer interface of a DPP-IV family of prolyl dipeptidases sufficient to prevent dimerization of members of the DPP-IV family of prolyl dipeptidases.


SEQUENCE LISTING (22 sequences)

SEQ ID NO: 1 - DNA, 25 bp, Artificial Sequence
  Description of Artificial Sequence  Synthetic primer
agcacaccaagaaatatatacccac

SEQ ID NO: 2 - DNA, 25 bp, Artificial Sequence
  Description of Artificial Sequence  Synthetic primer
gtgggtatatatttcttggtgtgct

SEQ ID NO: 3 - DNA, 25 bp, Artificial Sequence
  Description of Artificial Sequence  Synthetic primer
agcacaccaagctatatatacccac

SEQ ID NO: 4 - DNA, 25 bp, Artificial Sequence
  Description of Artificial Sequence  Synthetic primer
gtgggtatatatagcttggtgtgct

SEQ ID NO: 5 - protein, 54 aa, Homo sapiens
FQQSAQISKALVDVGVDFQAMWYTDEDHGIASSTAHQHIYTHMSHFIKQCFSLP

SEQ ID NO: 6 - protein, 27 aa, Homo sapiens
PLIEYSFYSDESLQYPKTVRVPYPKAG

SEQ ID NO: 7 - protein, 102 aa, Homo sapiens
VYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 8 - protein, 102 aa, Homo sapiens
VYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 9 - protein, 115 aa, Homo sapiens
AFSERYLGLHGLDNRAYEMTKVAHRVSALEEQQFLIIHPTADEKIHFQHTAELITQLIRGKANYSLQIYPDESHYFTSSSLKQHLYRSIINFFVECFRIQDKLPTVTAKEDEEED

SEQ ID NO: 10 - protein, 109 aa, Homo sapiens
GYTERYMGHPDQNEQGYYLGSVAMQAEKFPSEPNRLLLLHGFLDENVHFAHTSILLSFLVRAGKPYDLQIYPQERHSIRVPESGEHYELHLLHYLQENLGSRIAALKVI

SEQ ID NO: 11 - protein, 99 aa, Homo sapiens
GYTERYMDVPENNQHGYEAGSVALHVEKLPNEPNRLLILHGFLDENVHFFHTNFLVSQLIRAGKPYQLQIYPNERHSIRCPESGEHYEVTLLHFLQEYL

SEQ ID NO: 12 - protein, 115 aa, Homo sapiens; identical to SEQ ID NO: 9

SEQ ID NO: 13 - protein, 27 aa, Homo sapiens
PLIEYSFYSDESLQYPKTVRVPYPKAG

SEQ ID NO: 14 - protein, 25 aa, Homo sapiens
PVIAYSYYGDEQYPRTINIPYPKAG

SEQ ID NO: 15 - protein, 25 aa, Homo sapiens
PIMELPTYTGSIYPTVKPYHYPKAG

SEQ ID NO: 16 - protein, 24 aa, Homo sapiens
EIIHVTSPMLETRRADSFRYPKTG

SEQ ID NO: 17 - protein, 24 aa, Homo sapiens
EVIHVPSPALEERKTDSYRYPRTG

SEQ ID NO: 18 - protein, 25 aa, Homo sapiens
PIMELPTYTGSIYPTVKPYHYPKAG

SEQ ID NO: 19 - DNA, 25 bp, Artificial Sequence
  Description of Artificial Sequence  Synthetic primer
cgggatccatgcccatggggtctct

SEQ ID NO: 20 - DNA, 23 bp, Artificial Sequence
  Description of Artificial Sequence  Synthetic primer
ccgctcgagccgaggcaggaagc

SEQ ID NO: 21 - DNA, 25 bp, Artificial Sequence
  Description of Artificial Sequence  Synthetic primer
ccgctcgagaaaaacttacactcta

SEQ ID NO: 22 - DNA, 29 bp, Artificial Sequence
  Description of Artificial Sequence  Synthetic primer
gcgtcgacctaaggtaaagagaaacattg